(19) United States
(12) Patent Application Publication
(10) Pub. No.: US 2016/0137505 A1
(43) Pub. Date: May 19, 2016

(54) METHOD FOR SEPARATING METALLIC SINGLE-WALLED CARBON NANOTUBE FROM SEMICONDUCTIVE SINGLE-WALLED CARBON NANOTUBE

(71) Applicant: KYUSHU UNIVERSITY, NATIONAL UNIVERSITY CORPORATION, Fukuoka-shi, Fukuo (JP)

(72) Inventors: Naotoshi NAKASHIMA, Fukuoka (JP); Yasuro NIIDOME, Fukuoka (JP); Yuichi KATO, Fukuoka (JP)

(73) Assignee: KYUSHU UNIVERSITY, NATIONAL UNIVERSITY CORPORATION, Fukuoka-shi, Fukuoka (JP)

(21) Appl. No.: 14/772,526

(22) PCT Filed: Mar. 10, 2014
(86) PCT No.: PCT/JP2014/056214

§ 371 Date: Nov. 30, 2015

(30) Foreign Application Priority Data
Mar. 8, 2013 (JP) 2013-046851

(51) Int. Cl. C01B 31/02; C07D 475/14

(52) U.S. Cl. CPC C01B 31/0266; C07D 475/14

(57) ABSTRACT

Provided is a novel method for efficiently separating a metallic SWNT and a semiconducting SWNT from single-walled carbon nanotubes (SWNTs). The present invention is a method for separating a metallic SWNT and a semiconducting SWNT from SWNTs, said method comprising: dispersing the SWNTs in a solution containing a low-molecular-weight compound having an alkyl chain moiety for exhibiting solubility in a solvent and an aromatic-ring-containing moiety for interacting with the SWNTs; and separating the dispersion into a solution fraction and a solid fraction.

### TECHNICAL FIELD

The present invention relates to a method for efficiently separating metallic single-walled carbon nanotubes and semiconducting single-walled carbon nanotubes from single-walled carbon nanotubes (hereinafter, CNTs) containing both of them.

### BACKGROUND ART

A carbon nanotube (CNT) is a tubular material with a diameter of several nm to several tens of nm made by rolling a graphene sheet (a layer of six-membered carbon rings) into a cylinder, which is drawing attention as a superior nanomaterial that has thermal and chemical stability, mechanical strength, electron conductivity, thermal conductivity and spectral characteristics that extend to the near-infrared region.

Furthermore, CNTs include single-walled CNTs (hereinafter, SWNT) having a single layer of the above-described graphene sheet, double-walled CNTs (hereinafter, DWNTs) having two layers of the above-described graphene sheets, and a multi-walled CNT (hereinafter, MWNTs) having two or more layers of the above-described graphene sheets. In particular, SWNTs are attracting attention for its remarkable quantum effect.

SWNTs can be classified into armchair types, zigzag types and chiral types according to their difference in chirality (helicity), and their electric characteristics (band gap, electron level, etc.) are known to vary depending on the chiral angle along with a change in the structure such as the diameter. It is known that the armchair-type carbon nanotubes have metallic electric characteristics, while carbon nanotubes with other chiral angles may have semiconducting electric characteristics. The band gap of the SWNTs having such semiconducting electric characteristics (hereinafter, “semiconducting SWNTs”) vary depending on chirality. Utilizing such physical properties, semiconducting SWNTs are expected as materials for a high-performance transistor, an ultrashort pulse generator, an optical switch and the like. On the other hand, single-walled carbon nanotubes having metallic electric characteristics (hereinafter, “metallic SWNTs”) are expected as a replacement for a transparent conductive material that uses a rare metal so as to be applied to a transparent electrode for a liquid crystal display or a solar cell panel.

Now, SWNTs can be synthesized by various methods including a laser vaporization method, an arc discharge method and a chemical vapor deposition method (CVD method). Under the existing conditions, however, the metallic SWNTs and the semiconducting SWNTs are obtained only in a form of a mixture using any of these synthesis methods.

Therefore, development of a technique for separating semiconducting SWNTs and metallic SWNTs has been encouraged.

Conventional techniques, however, are associated with problems, such as requirement of multiple steps and poor yield of the SWNTs. These problems are major obstacles to practical (industrial) use. Conventional techniques also have problems, such as difficulty in the removal of the dispersant used for separation, and short length of the separated SWNTs. These problems cause increase in the resistivity upon the above-described application of the metallic SWNTs, while these problems cause deterioration in the transistor performance upon application of the semiconducting SWNTs.

One specific example of the above-mentioned conventional techniques is a method in which CNTs dispersed with a surfactant are subjected to dielectrophoretic between microelectrodes (Non-patent Document 1). There is also a method in which a solution of SWNTs dispersed with a soluble flavin derivative is prepared, to which a surfactant is added to give flavin derivative-dispersed SWNTs having specific chirality and surfactant-dispersed SWNTs having specific chirality and then the surfactant-dispersed SWNTs are removed by a salting-out method for separation (Non-patent Document 2).

Moreover, there are also a method in which a mixture of semiconducting SWNTs and metallic SWNTs is dispersed in a liquid so as to allow the metallic SWNTs to selectively bind to particles and then the metallic SWNTs bound to the particles are removed, thereby separating the semiconducting SWNTs (Patent Document 1), a method in which pH or ionic strength of a solution of SWNTs dispersed with a surfactant is adjusted so as to cause protonations at varying levels depending on the types of the SWNTs, which is subjected to an electric field so as to separate the metallic and semiconducting types (Patent Document 9), and a method, in which SWNTs dispersed with nucleic acid molecules are separated by ion-exchange chromatography (Patent Document 5).

Furthermore, there is a method in which SWNTs dispersed with a surfactant is separated into metallic SWNTs and semiconducting SWNTs by density-gradient ultracentrifugation (Non-patent Document 3).

In addition, there is a method, in which SWNT-containing gel obtained by soaking SWNTs dispersed with a surfactant into gel is used to separate the metallic SWNTs and the semiconducting SWNTs by a physical separating procedure (Patent Documents 6-8, and Non-patent Documents 4 and 5).

These methods proceed in two stages, namely, a step of dispersing SWNTs with a dispersant and a step of separating the SWNTs, requiring a multiple-stage process, which is difficult to be applied to industrial use. Moreover, since high-power ultrasonic irradiation and ultracentrifugation are employed in the first step, there are problems of poor yield of the SWNTs and short length of the separated SWNTs.

As other conventional methods, for example, there is a method in which semiconducting SWNTs are selectively burned with hydrogen peroxide (Non-patent Document 6). There are also a method in which SWNTs are treated with a nitronium-ion-containing solution and then subjected to filtration and heat treatment to remove the metallic SWNTs being contained in the SWNTs, thereby obtaining the semiconducting SWNTs (Patent Document 2), a method in which sulfuric acid and nitric acid are used (Patent Document 3), a method in which an electric field is applied so as to selectively move and separate the SWNTs, thereby obtaining the semiconducting SWNTs in the narrowed electric conductivity range (Patent Document 4).

Although these methods allow dispersion and separation to take place in a single step, they have problems in that only either the semiconducting SWNTs or the metallic SWNTs can be obtained, in that the recovery rate of the SWNTs is poor, and in that the length of the separated SWNTs is short, leading to defects.

As other conventional methods, there are, for example, methods in which a polyfluorene derivative (Non-patent Documents 7-10), polyalkylcarbazole (Non-patent Document 11) or polyalkylthiophene (Non-patent Document 12) is used to selectively disperse the semiconducting SWNTs in an organic solvent. These methods include a single working step and do not require ultracentrifugation upon separation. However, there is a problem of poor yield of the dispersed semiconducting SWNTs. In addition, since the dispersant is a polymer, there is a problem of significant difficulty in removal thereof after the separation due to strong adsorption with the SWNTs.

On the other hand, as methods for removing a dispersant after the separation, for example, there are a method in which an oligomer of a fluorene derivative is synthesized to disperse the SWNTs (Non-patent Document 13), a method in which the structure of the polymer is altered by photoreaction to reduce the adsorption power to the SWNTs (Non-patent Document 14), and a method in which a foldamer is used to alter the solvent condition so as to reduce the adsorption power to the SWNTs (Non-patent Document 15). These methods, however, have problems in that they do not allow selective dispersibility between the semiconducting SWNTs and the metallic SWNTs, and in that the yield of the dispersed semiconducting SWNTs is poor.

### PRIOR ART DOCUMENTS

### Patent Documents

Patent Document 1: Japanese Unexamined Patent Application Publication No. 2007-31238
Patent Document 2: Japanese Unexamined Patent Application Publication No. 2005-325020
Patent Document 3: Japanese Unexamined Patent Application Publication No. 2005-194180
Patent Document 4: Japanese Unexamined Patent Application Publication No. 2005-104750
Patent Document 5: Japanese Unexamined Patent Application Publication No. 2006-512276
Patent Document 6: International Publication No. 2009/75293
Patent Document 7: Japanese Unexamined Patent Application Publication No. 2011-168417
Patent Document 8: Japanese Unexamined Patent Application Publication No. 2011-195431
Patent Document 9: Japanese Unexamined Patent Application Publication No. 2005-527455

### Non-Patent Documents

Non-patent Document 1: Krupke, R.; Linden, S.; Rapp, M.; Hennrich, F. Adv. Mater. 2006, 18, 1468-1470.
Non-patent Document 2: Ju, S.-Y.; Doll, J.; Sharma, I.; Papadimitrakopoulos, F. Nature nanotechnology 2008, 3, 356-362.
Non-patent Document 3: Arnold, M. S.; Green, A. a; Hulvat, J. F.; Stupp, S. I.; Hersam, M. C. Nat. Nanotechnol. 2006, 1, 60-65.
Non-patent Document 4: Tanaka, T.; Jin, H.; Miyata, Y.; Fujii, S.; Suga, H.; Naitoh, Y.; Minari, T.; Miyadera, T.; Tsukagoshi, K.; Kataura, H. Nano letters 2009, 9, 1497-500.
Non-patent Document 5: Liu, H.; Nishide, D.; Tanaka, T.; Kataura, H. Nature communications 2011, 2, 309.
Non-patent Document 6: Miyata, Y.; Maniwa, Y.; Kataura, H. J. Phys. Chem. B 2006, 110, 25-29.
Non-patent Document 7: Nish, A.; Hwang, J.-Y.; Doig, J.; Nicholas, R. J. Nat. Nanotechnol. 2007, 2, 640-646.
Non-patent Document 8: Chen, F.; Wang, B.; Chen, Y.; Li, L.-J. Nano letters 2007, 7, 3013-3017.
Non-patent Document 9: Ozawa, H.; Fujigaya, T.; Niidome, Y.; Hotta, N.; Fujiki, M.; Nakashima, N. J. Am. Chem. Soc. 2011, 133, 2651-2657.
Non-patent Document 10: Akazaki, K.; Toshimitsu, F.; Ozawa, H.; Fujigaya, T.; Nakashima, N. J. Am. Chem. Soc. 2012, 134, 12700-12707.
Non-patent Document 11: Lemasson, F. A.; Strunk, T.; Gerstel, P.; Hennrich, F.; Lebedkin, S.; Barner-Kowollik, C.; Wenzel, W.; Kappes, M. M.; Mayor, M. J. Am. Chem. Soc. 2011, 133, 652-655.
Non-patent Document 12: Lee, H. W.; Yoon, Y.; Park, S.; Oh, J. H.; Hong, S.; Liyanage, L. S.; Wang, H.; Morishita, S.; Patil, N.; Park, Y. J.; Park, J. J.; Spakowitz, A.; Galli, G.; Gygi, F.; Wong, P. H.-S.; Tok, J. B.-H.; Kim, J. M.; Bao, Z. Nature communications 2011, 2, 541.
Non-patent Document 13: Berton, N.; Lemasson, F.; Hennrich, F.; Kappes, M. M.; Mayor, M. Chem. Commun. 2012, 48, 2516-2518.
Non-patent Document 14: Umeyama, T.; Kawabata, K.; Tezuka, N.; Matano, Y.; Miyato, Y.; Matsushige, K.; Tsujimoto, M.; Isoda, S.; Takano, M.; Imahori, H. Chemical communications (Cambridge, England) 2010, 46, 5969-5971.
Non-patent Document 15: Zhang, Z.; Che, Y.; Smaldone, R. a; Xu, M.; Bunes, B. R.; Moore, J. S.; Zang, L. J. Am. Chem. Soc. 2010, 132, 14113-14117.

### SUMMARY OF THE INVENTION

### Problems to be Solved by the Invention

The above-described conventional techniques have problems in that they require a multiple-stage process and in that the yield of SWNTs is poor, where these problems are major obstacles to industrial use. Moreover, conventional techniques also have problems in that removal of the dispersant used for separation is difficult and in that the length of the separated SWNTs is short. These problems cause increase in the resistivity upon the above-described application of the metallic SWNTs while these problems cause deterioration in the transistor performance upon application of the semiconducting SWNTs.

Hence, the problem that the present invention intends to solve is to provide a novel method for efficiently separating metallic SWNTs and semiconducting SWNTs from SWNTs, which can solve the above-described problems.

### Means for Solving the Problems

The present inventors have gone through keen examination to solve the above-described problem. As a result, they found that SWNTs can be separated into metallic SWNTs and semiconducting SWNTs by selectively dispersing (solubilizing) the semiconducting SWNTs with a low-molecular-weight compound. They also found that the low-molecular-weight compound can be removed from the SWNTs by washing with a solvent and that the SWNTs can be redispersed with other surfactant or the like. Consequently, the present invention was completed.

A “low-molecular-weight compound” as used herein refers to a low-molecular-weight compound having an alkyl chain moiety for exhibiting solubility in a solvent and an aromatic-ring-containing moiety for interacting with SWNTs. For example, a flavin derivative soluble in an organic solvent is preferable. Specific steps include, for example, but not limited to, adding a flavin derivative and SWNTs in an organic solvent, dispersing the SWNTs by ultrasonic irradiation and subjecting this dispersion solution to centrifugation, thereby obtaining the supernatant (solution fraction) thereof as a solution having the dispersed semiconducting SWNTs. Meanwhile, the metallic SWNTs can be obtained as a precipitate (solid fraction) containing the same.

Thus, the present invention is as follows.

(1) A method for separating a metallic SWNT and a semiconducting SWNT from SWNTs, said method comprising the steps of: dispersing the SWNTs in a solution containing a low-molecular-weight compound having an alkyl chain moiety for exhibiting solubility in a solvent and an aromatic-ring-containing moiety for interacting with the SWNTs; and separating said dispersion solution into a solution fraction and a solid fraction.

Here, the above-mentioned low-molecular-weight compound may be any low-molecular-weight compound with chiral selectivity, for example, but not particularly limited to, those containing a flavin derivative, specifically, those containing 10-dodecyl-7,8-dimethyl-10H-benzo[g]pteridine-2,4-dione (chemical structural formula shown as structural formula (1) below) and/or 10-octadecyl-7,8-dimethyl-10H-benzo[g]pteridine-2,4-dione.

Preferably, in the separation method according to (1) above, the solubilized semiconducting SWNTs are contained in the above-described solution fraction, while the metallic SWNTs are contained in the precipitated fraction.

In the separation method according to (1) above, the dispersion is carried out, for example, by stirring, shaking, ball milling or ultrasonic irradiation, while the separation is carried out, for example, by settling, filtration, membrane separation, centrifugation or ultracentrifugation.

The separation method according to (1) above may be, for example, a method that further comprises the steps of: collecting the semiconducting SWNTs from the above-described solution fraction; and/or collecting the metallic SWNTs from the above-described solid fraction.

(2) An agent for separating metallic SWNTs and semiconducting SWNTs, the agent comprising a low-molecular-weight compound having an alkyl chain moiety for exhibiting solubility in a solvent and an aromatic-ring-containing moiety for interacting with the SWNTs.

Here, the above-mentioned low-molecular-weight compound may be any low-molecular-weight compound with chiral selectivity, for example, but not particularly limited to, those containing a flavin derivative, specifically, those containing 10-dodecyl-7,8-dimethyl-10H-benzo[g]pteridine-2,4-dione (chemical structural formula shown as structural formula (1) below) and/or 10-octadecyl-7,8-dimethyl-10H-benzo[g]pteridine-2,4-dione.

### Effect of the Invention

According to the present invention, SWNTs separated into semiconducting SWNTs and metallic SWNTs can be obtained in a single working step with an inexpensive equipment. Moreover, SWNTs longer than those obtained by the conventional techniques can be obtained at a high recovery rate. Furthermore, since the dispersant can be removed after the separation, application to a wide range of usage is not restrained by separation.

### BRIEF DESCRIPTION OF DRAWINGS

FIG. 1 A diagram showing an absorption spectrum (solid line) of SWNTs dispersed in toluene with one of flavin derivatives, 10-dodecyl-7,8-dimethyl-10H-benzo[g]pteridine-2,4-dione (10-Dodecyl-7,8-dimethyl-10H-benzo[g]pteridine-2,4-dione) (hereinafter, sometimes referred to as FC12 or dmC12) and an absorption spectrum (dotted line) of FC12. In FIG. 1, absorption of the metallic SWNTs appears at 400-600 nm while the absorption spectrum of SWNTs dispersed with FC12 shows no absorption peak at 500-600 nm. Absorption at 950-1600 nm results from E^{s}₁₁ of the semiconducting SWNTs. Absorption at 600-900 nm results from E^{s}₂₂ of the semiconducting SWNTs.

FIG. 2 A view showing a photoluminescence spectrum of SWNTs dispersed in toluene with FC12.

FIG. 3 A diagram showing a Raman spectrum (solid line) of SWNTs dispersed in toluene with FC12 and a Raman spectrum of SWNTs dispersed in water.

FIG. 4 A view showing an AFM image of SWNTs dispersed in toluene with FC12.

FIG. 5 A diagram showing distribution of lengths of SWNTs dispersed in toluene with FC12 determined based on the AFM image. The average length was 1.1 p.m.

FIG. 6 A diagram showing an absorption spectrum (solid line) of a solution obtained by recollecting the SWNTs dispersed in toluene with FC12 and redispersing them using sodium cholate and an absorption spectrum (dotted line, control) of a solution of SWNTs dispersed using sodium cholate. The relatively low absorbance of 450-600 nm represents the decrease in the metallic SWNTs.

FIG. 7 A diagram showing absorption spectra of SWNTs dispersed in toluene with FC12. Cases with various centrifugal accelerations.

FIG. 8 A diagram showing an absorption spectrum of SWNTs dispersed in o-xylene with FC12.

FIG. 9 A diagram showing an absorption spectrum of SWNTs dispersed in p-xylene with FC12.

FIG. 10 A diagram showing absorption spectra of SWNTs dispersed in o-dichlorobenzene with FC12.

FIG. 11 Diagrams showing the results of measurements of average migration lengths of dmC12 (FC12) on the semiconducting SWNTs with and without imide hydrogen (—NH—) at position 3 of 10-dodecyl-7,8-dimethyl-10H-benzo[g]pteridine-2,4-dione (dmC12 or FC12), i.e., one of flavin derivatives. This measurement was carried out by MD (Molecular Dynamics) following structural optimization with the molecular mechanics calculation (MM).

FIG. 12 With respect to a dimer of 10-dodecyl-7,8-dimethyl-10H-benzo[g]pteridine-2,4-dione (dmC12 or FC12), i.e., one of flavin derivatives, diagrams showing the results of measurements of average migration lengths of dmC12 (FC12) on the respective SWNTs (semiconducting SWNTs and metallic SWNTs). This measurement was carried out by MD (Molecular Dynamics) following structural optimization with the molecular mechanics calculation (MM).

FIG. 13 Diagrams showing absorption spectra (UV-vis-NIR) and photoluminescence spectra (2D-PL) of the SWNTs dispersed in toluene with one of flavin derivatives, 10-dodecyl-7,8-dimethyl-10H-benzo[g]pteridine-2,4-dione (dmC12 or FC12) or 10-octadecyl-7,8-dimethyl-10H-benzo[g]pteridine-2,4-dione (hereinafter, sometimes referred to as dmC18).

### MODES FOR CARRYING OUT THE INVENTION

Hereinafter, the present invention will be described in detail. The scope of the present invention is not restricted to these descriptions, and can appropriately be modified and carried out apart from the following examples without departing from the spirit of the present invention.

The present specification incorporates the entire content of the specification of Japanese Patent Application No. 2013-046851 (filed on Mar. 18, 2013) based on which the present application claims priority. In addition, all of the publications, for example, prior art documents and publications, patent publications and other patent documents, cited herein are incorporated herein by reference.

As already mentioned above, herein, a single-walled carbon nanotube is referred to as a “SWNT”, a semiconducting single-walled carbon nanotube is referred to as a “semiconducting SWNT” and a metallic single-walled carbon nanotube is referred to as a “metallic SWNT”.

As already mentioned above, the present invention is a method for separating a metallic SWNT and a semiconducting SWNT from SWNTs containing a mixture of the metallic SWNTs and the semiconducting SWNT.

Specifically, this separation method comprises the steps of: dispersing SWNTs in a solution containing a low-molecular-weight compound having predetermined physical property and structure; and separating said dispersion solution into a solution fraction and a solid fraction. According to this method, the solubilized semiconducting SWNTs are contained (separated) in the solution fraction while the metallic SWNTs are contained (separated) in the solid fraction. This separation method may also comprise the step of recovering the semiconducting SWNTs from the above-mentioned solution fraction or recovering the metallic SWNTs from the above-mentioned solid fraction.

According to the separation method of the present invention, examples of SWNTs targeted by the above-described separation include those that are synthesized by the HiPCO method, CoMocat method, ACCVD method, arc discharge method, laser ablation method or the like.

According to the separation method of the present invention, an example of the low-molecular-weight compound used as the dispersant includes a low-molecular-weight compound having an alkyl chain moiety for exhibiting solubility in the solvent and an aromatic-ring-containing moiety for interacting with the SWNTs. This low-molecular-weight compound may be any low-molecular-weight compound having chiral selectivity, for example, but not particularly limited to, a flavin derivative, particularly preferably a flavin derivative soluble in an organic solvent. Specifically, this flavin derivative is preferably, for example, but not limited to, 10-dodecyl-7,8-dimethyl-10H-benzo[g]pteridine-2,4-dione (FC12 or dmC12) or the like represented by the following Structural formula (1). For example, a moiety of an alkyl group expressed as —C₁₂H₂₅ in the following structural formula may have variation in the lengths of the alkyl group within a range that allows solubility in the solvent. Specifically, preferable examples includes those with an alkyl group expressed as —CₘH₂ₘ₊₁ (wherein, m is preferably an integer of 5-25, and more preferably an integer of 10-20). In particular, a preferable example includes a flavin derivative wherein “m” mentioned above is 18, namely, 10-octadecyl-7,8-dimethyl-10H-benzo[g]pteridine-2,4-dione (dmC18).

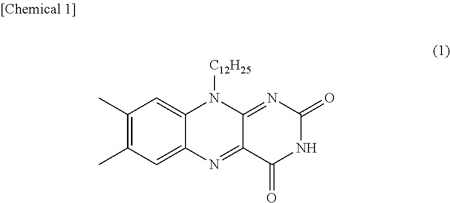

Here, the “methyl groups (—CH₃)” existing at positions 7 and 8 of the flavin derivative represented by Structural formula (1) above (including those having different lengths of alkyl groups “—CₘH₂ₘ₊₁” as described above) are considered to be important in that they can cause the CH-π interaction with the SWNTs targeted for separation (namely, attracting force acting between the hydrogen bound to carbon and the π electron system) and thus enhance solubility of the SWNTs (in particular, the semiconducting SWNTs).

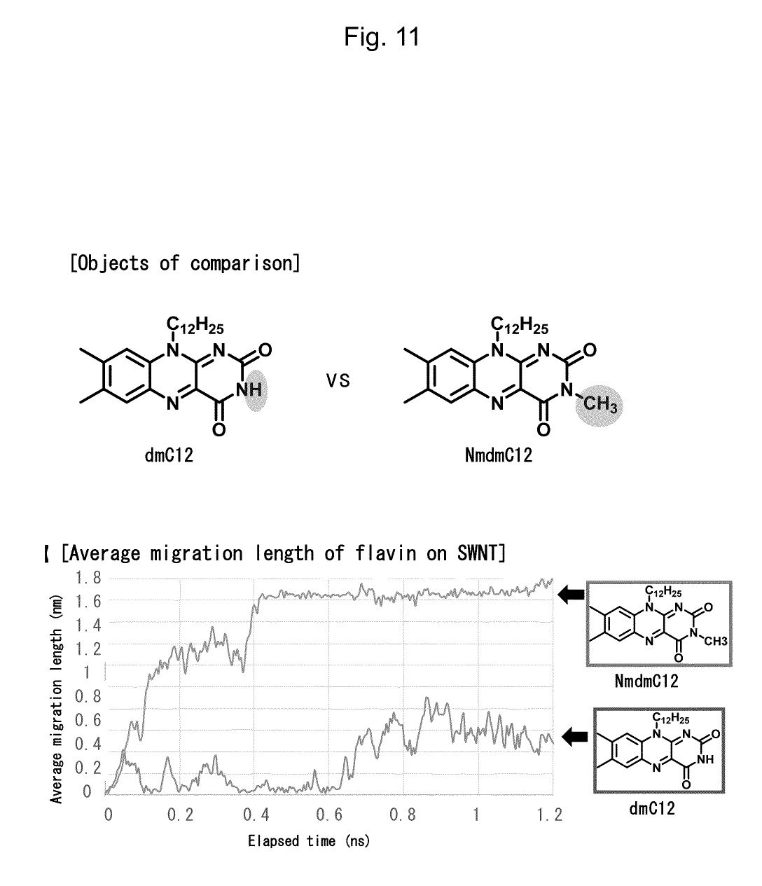

The “imide hydrogen (—NH—)” existing at position 3 of the flavin derivative is also considered to be important in that it is involved in the dimerization between the flavin derivatives used via hydrogen bonding, as a result of which more flavin derivatives can interact with (adsorb to) the SWNTs (in particular, the semiconducting SWNTs). This can also be understood from the fact that a flavin derivative with said imide hydrogen has shorter average migration length on the semiconducting SWNT, in other words, the interaction with (adsorptive property to) the semiconducting SWNTs is greater, as compared to a flavin derivative without said imide hydrogen as will be shown in Example 6 below and FIG. 11.

In addition, the above-described flavin derivative that interacts with (adsorbs to) the SWNTs has significant difference in the average migration length on the SWNTs, namely interaction with (adsorptive property to) the SWNTs, depending on whether the target of the interaction is semiconducting SWNTs or metallic SWNTs (average migration length: metallic SWNTs>semiconducting SWNTs) as will be shown in Example 7 below and FIG. 11 and thus it may be effective in increasing the solubility of the semiconducting SWNTs.

The solvent used with the separation method of the present invention may be any known organic solvent, for example, but not particularly limited to, benzene, toluene, xylene, ethylbenzene and the like, chlorobenzene, dichlorobenzene, chloromethylbenzene, bromobenzene and the like, naphthalene derivatives and the like, hexane, cyclohexane, THF, DMF and the like.

The procedure used upon dispersion (preparation of a dispersion solution) after the addition of a low-molecular-weight compound as the dispersant and SWNTs as the target of separation to the above-described solvent is not particularly limited and may be, for example, a procedure such as stirring, shaking, ball milling, ultrasonic irradiation (bath-type, probe-type, cup-type) or the like.

When this dispersion is carried out, for example, by ultrasonic irradiation, it is preferably carried out at a temperature condition of 5-80° C. (more preferably, 10-40° C.) for 5-720 minutes (more preferably, 10-180 minutes) although it is not particularly limited thereto.

The procedure for separating the dispersion solution into a solution fraction and a solid fraction after the dispersion described above, is not particularly limited, and may be, for example, a procedure such as settling, filtration, membrane separation, centrifugation, ultracentrifugation or the like.

The procedure for collecting the semiconducting SWNTs from the solution fraction after the separation, is not particularly limited, and may preferably be, for example, a procedure in which the solvent is removed by natural drying, with an evaporator or the like, or a procedure in which the semiconducting SWNTs are once aggregated by heating the solution fraction or dropping a good solvent for the dispersant and then subjected to filtration or membrane separation. In addition, the procedure for removing the dispersant is not particularly limited and may preferably be, for example, recrystallization (precipitation using change in the solubility by cooling), washing, sublimation, burning or the like.

On the other hand, the procedure for collecting the metallic semiconducting SWNTs from the solid fraction after the separation, is not particularly limited, and may preferably be, for example, a procedure such as filtration, membrane separation, centrifugation, ultracentrifugation or the like.

The present invention can also provide a dispersant that is capable of separating metallic SWNTs and semiconducting SWNTs from SWNTs containing a mixture of the metallic SWNTs and the semiconducting SWNTs.

Specifically, the dispersant contains a low-molecular-weight compound having an alkyl chain moiety for exhibiting solubility in the solvent and an aromatic-ring-containing moiety for interacting with single-walled carbon nanotubes as an active element, where the low-molecular-weight compound is not particularly limited as long as it has chiral selectivity.

Preferable examples of said low-molecular-weight compound include those that contain flavin derivatives. Specifically, those containing 10-dodecyl-7,8-dimethyl-10H-benzo[g]pteridine-2,4-dione (FC12 or dmC12) represented by Structural formula (1) shown above are more preferable although it is not particularly limited thereto. For example, a moiety of an alkyl group expressed as —C₁₂H₂₅ in the Structural formula (1) above may have variation in the length of the alkyl group within a range that exhibits solubility in the solvent. Specifically, preferable examples includes those with an alkyl group expressed as —CₘH₂ₘ₊₁ (wherein, m is preferably an integer of 5-25, and more preferably an integer of 10-20). In particular, a preferable example includes a flavin derivative wherein “m” mentioned above is 18, namely, 10-octadecyl-7,8-dimethyl-10H-benzo[g]pteridine-2,4-dione (dmC18).

The separating agent of the present invention may appropriately contain components other than the above-described low-molecular-weight compound as the active element, and is not particularly limited.

Hereinafter, the present invention will be described more specifically by means of examples although the present invention should not be limited thereto.

### Example 1

One of flavin derivatives, 10-dodecyl-7,8-dimethyl-10H-benzo[g]pteridine-2,4-dione (FC12 or dmC12; see Structural formula (1) below), was synthesized.

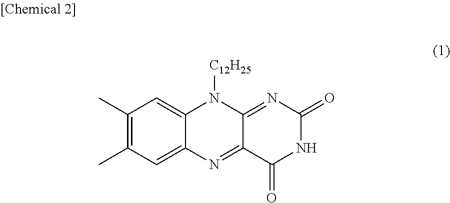

To toluene, FC12 and SWNTs synthesized by the HiPCO method, one of CVD methods, (which had already been removed of the catalyst with acid) were added to 0.6 mg/mL, and the resultant was subjected to ultrasonic irradiation for 3 hours with a bath-type ultrasonic irradiator (BRANSON5510). Thereafter, the dispersion liquid was centrifuged for 10 minutes under the condition of 10000×G at 25° C. with a cooling centrifuge (himac CF-15R) to collect the supernatant.

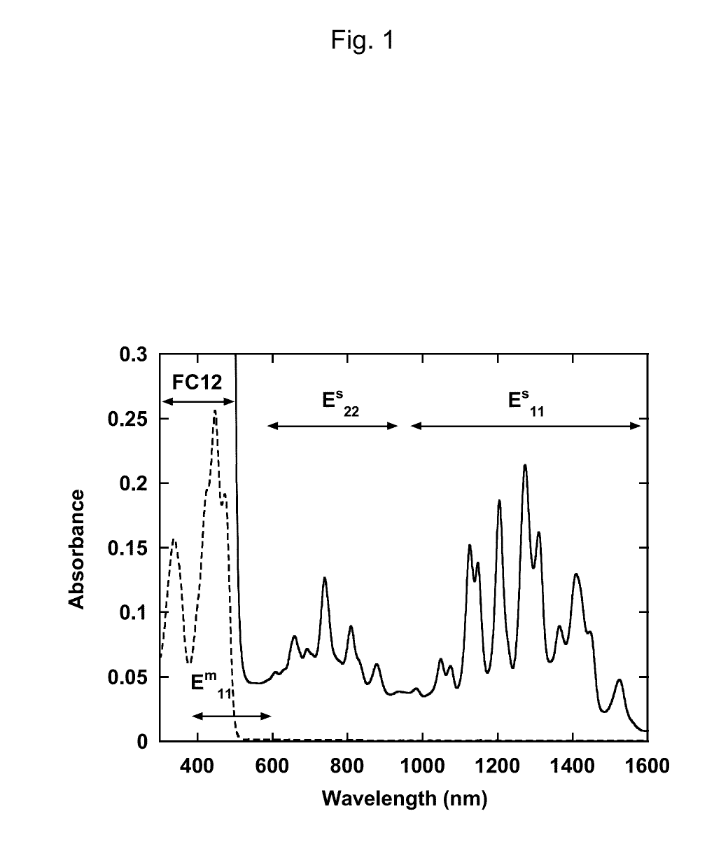

The absorption spectrum of the collected supernatant solution was measured. The solid line in FIG. 1 represents visible and near-infrared absorption spectra of the SWNTs dispersed in toluene. The light path length was 1 mm. In this diagram, the first bandgap of the semiconducting SWNTs can be seen between E^{s}₁₁ and E^{s}₂₂. From these two bands, it is obvious that FC12 isolated/dispersed the semiconducting SWNTs. The large visible absorbance at a wavelength of 500 nm or less results from FC12 (the dotted line in FIG. 1, 0.1 mg/mL FC12 toluene solution). The absence of a band of an isolated/dispersed SWNT at 500-550 nm and the low baseline indicate selective solubilization of the semiconducting SWNTs by FC12. The absorbance of the SWNTs of about 0.2 with respect to the light path length of 1 mm means that about 10 times of SWNTs had been isolated/dispersed as compared to the case of solubilization with a general surfactant (dodecyl sodium sulfate or sodium cholate) used in conventional techniques. The estimated concentration and yield of the SWNTs were about 0.05-0.12 mg/mL and 8-20%, respectively. The method for this estimation was carried out with reference to the paper of Shinohara et al. (Kuwahara, S.; Sugai, T.; Shinohara, H. Phys. Chem. Chem. Phys. 2009, 11, 1091-1097). The absorbance coefficient of SWNTs dispersed with dodecyl sodium sulfate containing both of the semiconducting SWNTs and the metallic SWNTs at 280 nm was 2.1±0.7×10⁻⁵ mg mL⁻¹ cm⁻¹ (supra, Kuwahara, S. et al., Chem. Chem. Phys. 2009). Judging from the shape of the absorption spectrum of the SWNTs, the absorbance coefficient in the visible region is about half of the absorbance coefficient at 280 nm (supra, Kuwahara, S. et al., Chem. Chem. Phys. 2009). Using this calculation to determine the yield based on the spectrum shown in FIG. 1, absorbance of 0.05 at 600 nm resulted 0.05 mg/mL while absorbance of 0.12 at 700 nm resulted 0.12 mg/mL.

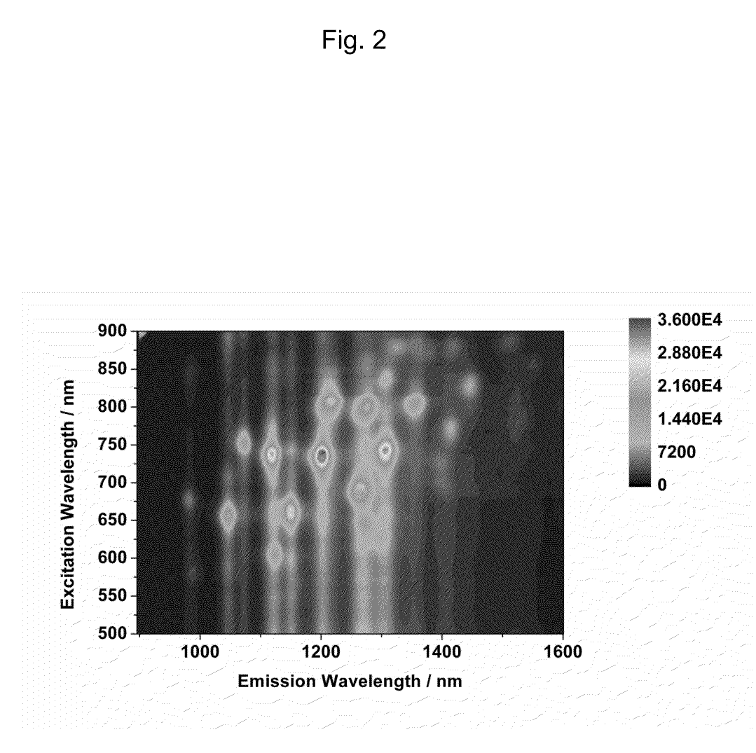

The photoluminescence of the collected supernatant solution was determined. FIG. 2 shows a two-dimensional photoluminescence map of SWNTs dispersed in toluene. As can be appreciated from the figure, the semiconducting SWNTs synthesized by the HiPCO method were solubilized almost evenly.

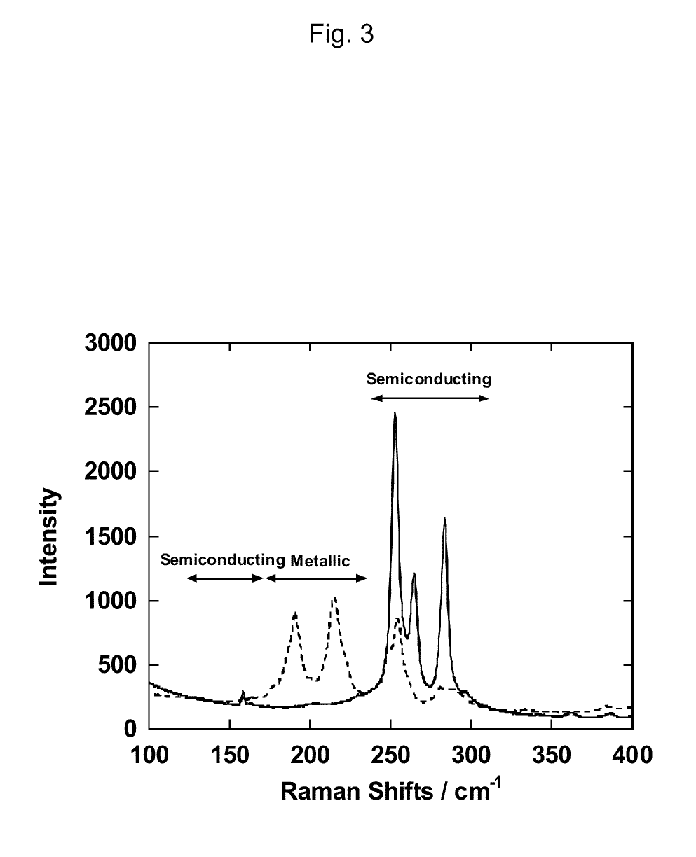

The collected supernatant solution was filtrated with a membrane filter (PTFE 0.1 μm (Millipore)) and washed with acetone. The Raman spectrum of the paper filter was determined. As a control, SWNTs synthesized by the HiPCO method was dispersed in water, filtrated with a membrane filter (HTTP 0.4 μm (Millipore)) to determine the Raman spectrum of the paper filter (excitation: wavelength 633 nm). The Raman spectra are shown in FIG. 3. The metallic/semiconducting ratio of the SWNTs synthesized by the HiPCO method (metallic SWNTs/semiconducting SWNTs) was difficult to determine from the absorption spectra (Miyata, Y.; Yanagi, K.; Maniwa, Y.; Kataura, H. J. Phys. Chem. C 2008, 112, 13187-13191). Accordingly, it was estimated from the peak area ratio of RBM of the Raman spectrum of the SWNTs at excitation of 633 nm (supra, Non-patent Document 4). Calculation based on the peak area of RBM of the Raman spectrum gave the above-described metallic/semiconducting ratio to be 97.4% on the basis of that of water-dispersed HiPCO.

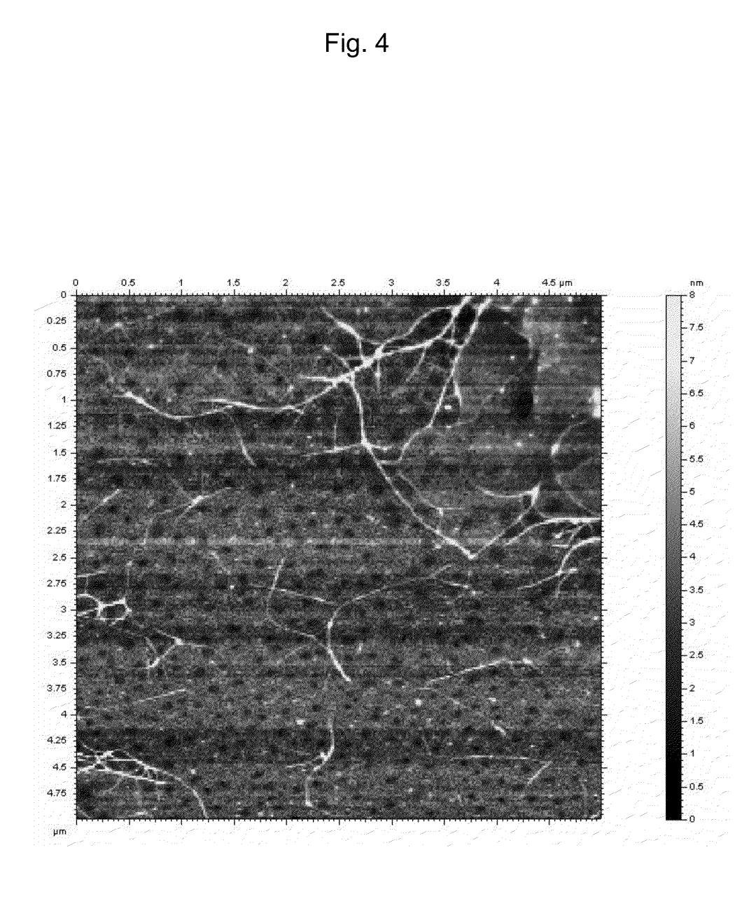

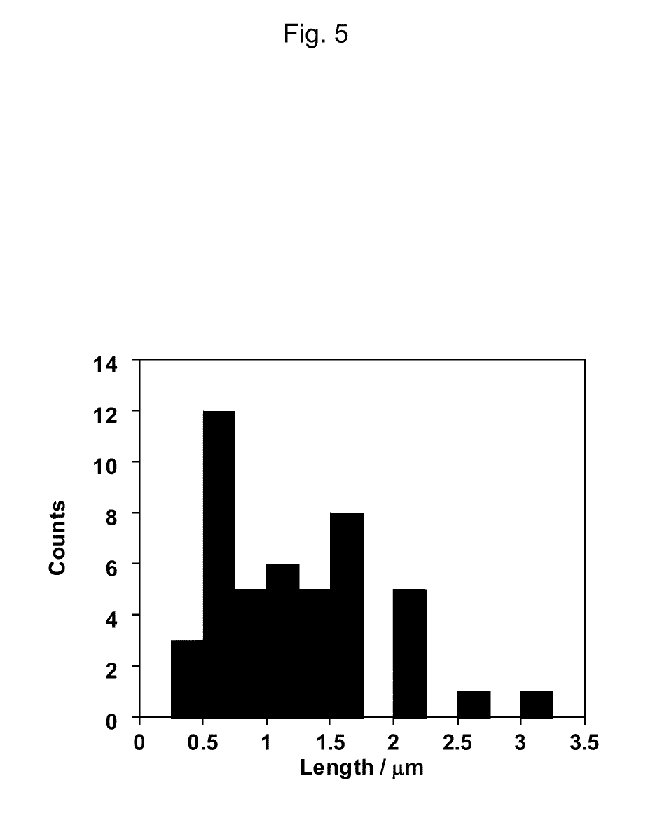

In order to examine the distribution of the SWNT lengths, an atomic force microscope (AFM) was used for determination. The collected supernatant solution was spin coated and washed with dichloromethane. FIG. 4 shows an AFM image of the SWNTs. The distribution of the lengths was determined from randomly selected 48 SWNT images. The distribution of the lengths is shown in FIG. 5. The average length was 1.1 μm. Since the average length of semiconducting SWNTs obtained by a conventional technique is about 0.4 μm, SWNTs with lengths that are about twice or three times longer were obtained. This owes to FC12 that can disperse a larger amount of SWNTs. Since dispersibility is good, dispersion can take place under mild conditions like ultrasonic irradiation using a bath-type ultrasonic irradiator, the shortening of the lengths of the SWNTs caused by ultrasonic wave can be avoided.

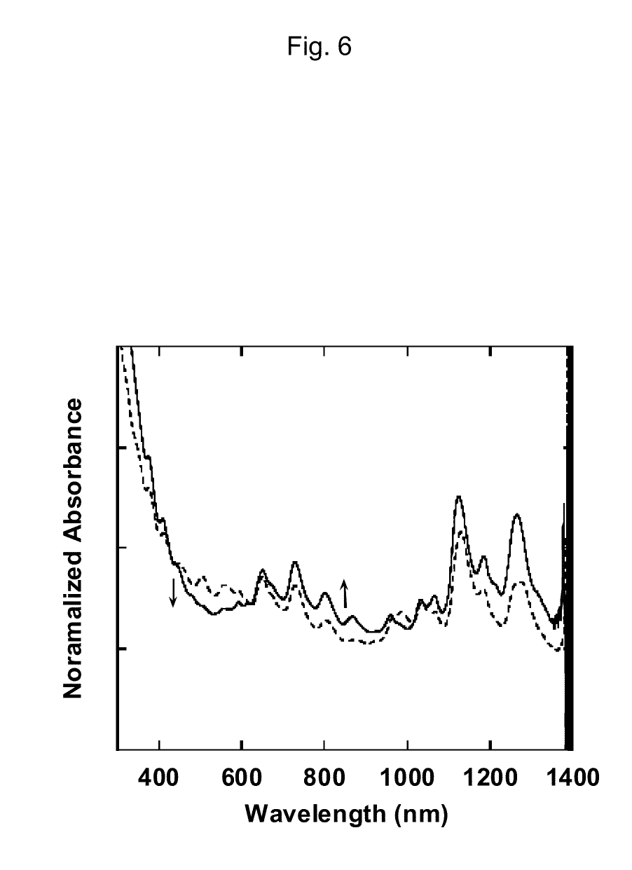

30 mL of the collected supernatant solution was cooled in a freezer (−5° C.) to precipitate and remove excessive FC12. Toluene was evaporated with an evaporator to precipitate the SWNTs on the side surface of the sample tube. These SWNTs were washed with 500 mL of acetone for 13 times. 10 mL of 1 wt % aqueous sodium cholate solution was added to that sample tube, which was subjected to ultrasonic irradiation using a bath-type ultrasonic irradiator for 3 hours and using a probe-type ultrasonic irradiator for 30 minutes while cooling in a water bath. The resulting solution was centrifuged once using an ultracentrifuge under the conditions of 120000×G and 25° C. to collect the supernatant. The absorption spectrum of the supernatant was determined. The result is represented by the solid line in FIG. 6. Meanwhile, as a control, a solution of SWNTs synthesized by the HiPCO method containing unseparated semiconducting and metallic SWNTs and dispersed with sodium cholate was centrifuged to collect the supernatant, whose absorption spectrum is represented by the dotted line in FIG. 6. Referring to the absorption spectrum, absorbance of the isolated/dispersed SWNTs was observed at 500-1400 nm. Since semiconducting SWNTs dispersed with a polyfluorene derivative or the like mentioned in the above-described conventional techniques cannot be washed, they cannot be dispersed in an aqueous solution with a surfactant. This result indicates that FC12 can be removed by washing since it is a low-molecular-weight compound. The relatively low absorbance of 450-600 nm as compared to the control shows that the metallic SWNTs were relatively decreased.

### Example 2

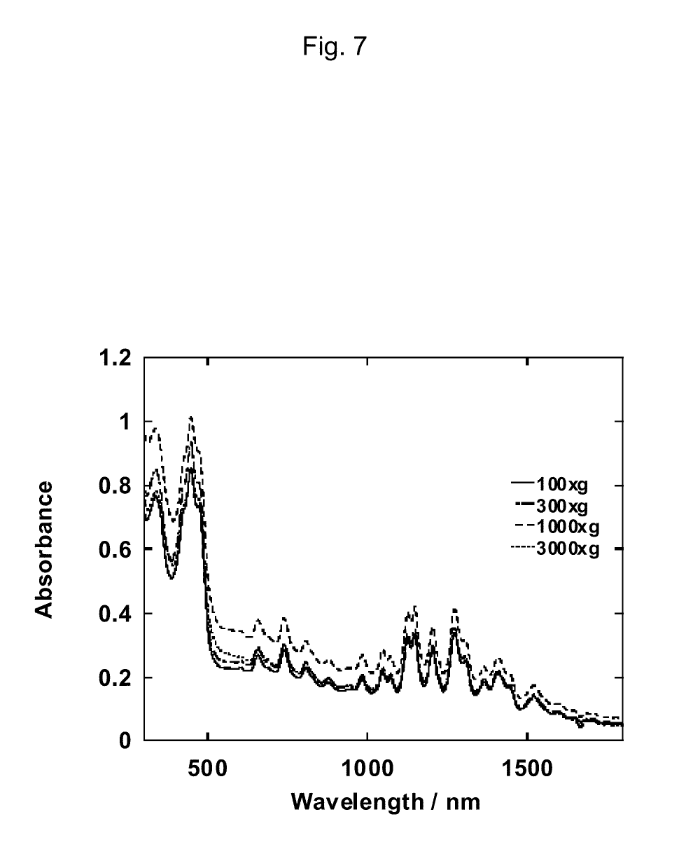

The centrifugal acceleration condition for the liquid of FC12 and SWNTs dispersed in toluene in Example 1 was altered to be 100×G, 500×G, 1000×G and 3000×G to determine the absorption spectra of the collected supernatants. The absorption spectra are shown in FIG. 7. Similar to the case of the condition in Example 1, i.e., 10000×G, selective solubilization of the semiconducting SWNTs were observed.

### Example 3

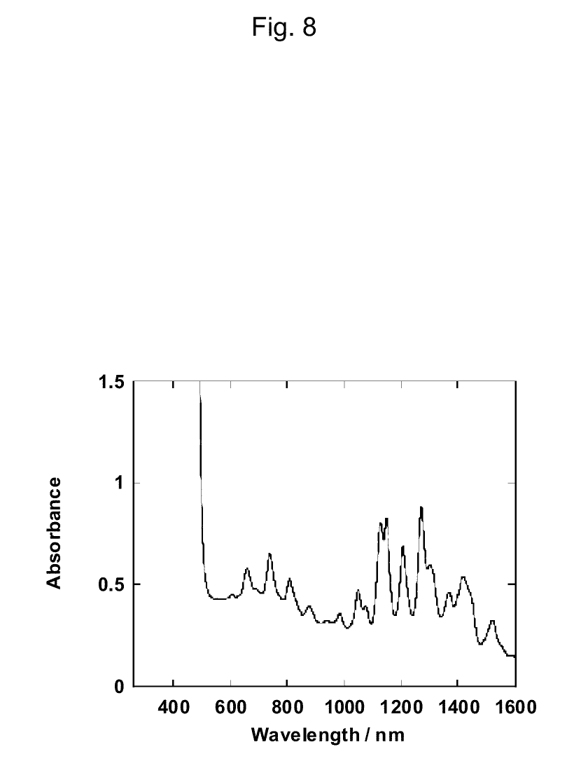

To o-xylene, FC12 and HiPCO SWNTs (SWNTs synthesized by the HiPCO method) (which had already been removed of the catalyst) were added, and the resultant was subjected to ultrasonic irradiation for 3 hours using a bath-type ultrasonic irradiator (BRANSON5510). Thereafter, the dispersion liquid was centrifuged with a cooling centrifuge (himac CF-15R) for 10 minutes under the condition of 10000×G at 25° C. to collect the supernatant. The absorption spectrum of the collected supernatant solution was measured (light path length: 1 cm). The absorption spectrum is shown in FIG. 8. Selective solubilization of the semiconducting SWNTs was observed.

### Example 4

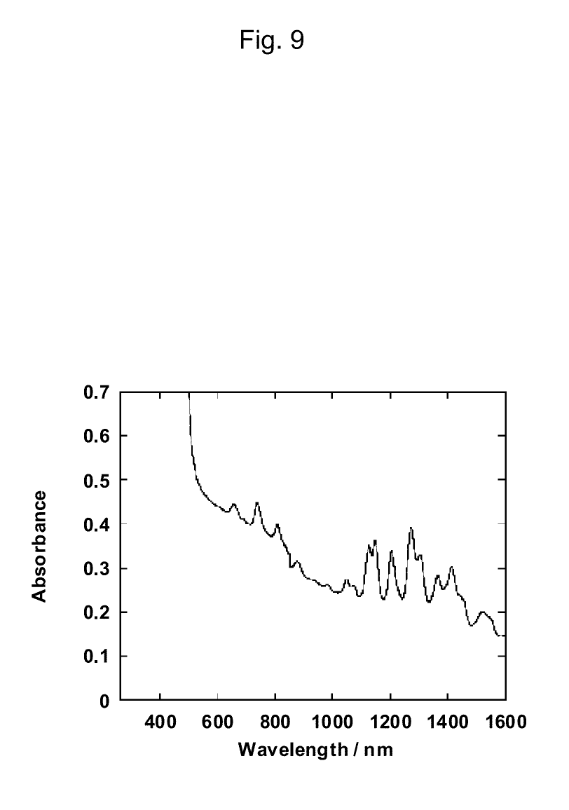

To p-xylene, FC12 and HiPCO SWNTs (which had already been removed of the catalyst) were added, and the resultant was subjected to ultrasonic irradiation for 3 hours using a bath-type ultrasonic irradiator (BRANSON5510). Thereafter, the dispersion liquid was centrifuged with a cooling centrifuge (himac CF-15R) for 10 minutes under the condition of 10000×G at 25° C. to collect the supernatant. The absorption spectrum of the collected supernatant solution was measured (light path length: 1 cm). The absorption spectrum is shown in FIG. 9. Selective solubilization of the semiconducting SWNTs was observed.

### Example 5

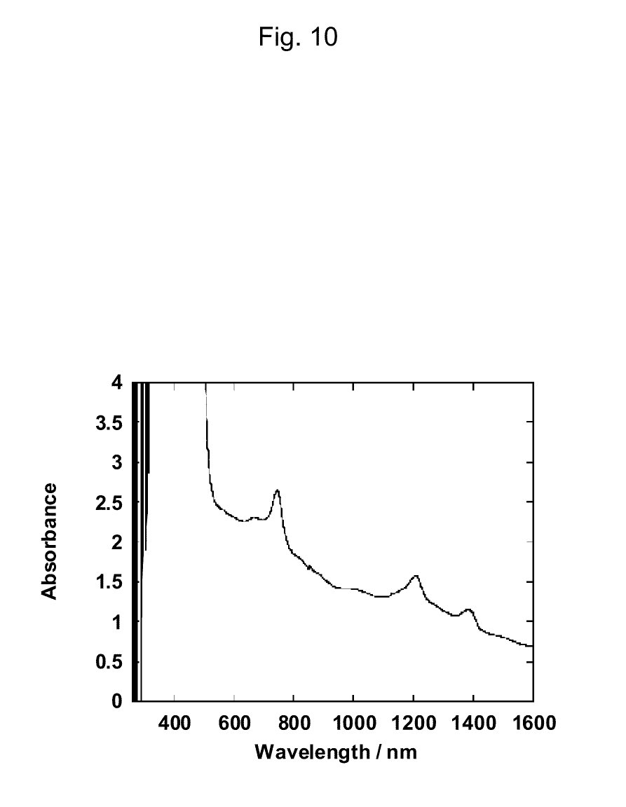

To o-dichlorobenzene, FC12 and HiPCO SWNTs (which had already been removed of the catalyst) were added, and the resultant was subjected to ultrasonic irradiation for 3 times with a bath-type ultrasonic irradiator (BRANSON5510). Thereafter, the dispersion liquid was centrifuged with a cooling centrifuge (himac CF-15R) for 10 minutes under the condition of 10000×G at 25° C. to collect the supernatant. The absorption spectrum of the collected supernatant solution was measured (light path length: 1 cm). The absorption spectrum is shown in FIG. 10. Selective solubilization of the semiconducting SWNTs was observed.

### Example 6

Interactions with (adsorptive properties to) semiconducting SWNTs were compared between the presence and the absence of imide hydrogen (—NH—) at position 3 of flavin derivatives (FC12 or dmC12), namely, the presence and the absence of hydrogen bonding between the flavin derivatives (the presence or the absence of dimerization capacity) by measuring the average migration lengths of the flavin derivatives on the semiconducting SWNTs according to the following measurement and experiment conditions. Specifically, the MD (Molecular Dynamics) was carried out following structural optimization with the molecular mechanics calculation (MM). The results are shown in FIG. 11.

<Measurement and Experiment Conditions>
MM (molecular mechanics calculation): SCHRODINGER MACROMODEL 9.6
MD (Molecular Dynamics): Desmond
Molecular force field: OPLS 2005
Solvent: toluene
Temperature: 300 K
Time: 1.2 ns
56 flavin derivative molecules were placed on (8,6) SWNT (semiconducting SWNT)

As can be appreciated from the results shown in FIG. 11, the flavin derivatives (dmC12) with imide hydrogen (—NH—) had a shorter average migration length on the semiconducting SWNTs and thus found to have greater interaction (greater adsorptive property) with the semiconducting SWNTs.

### Example 7

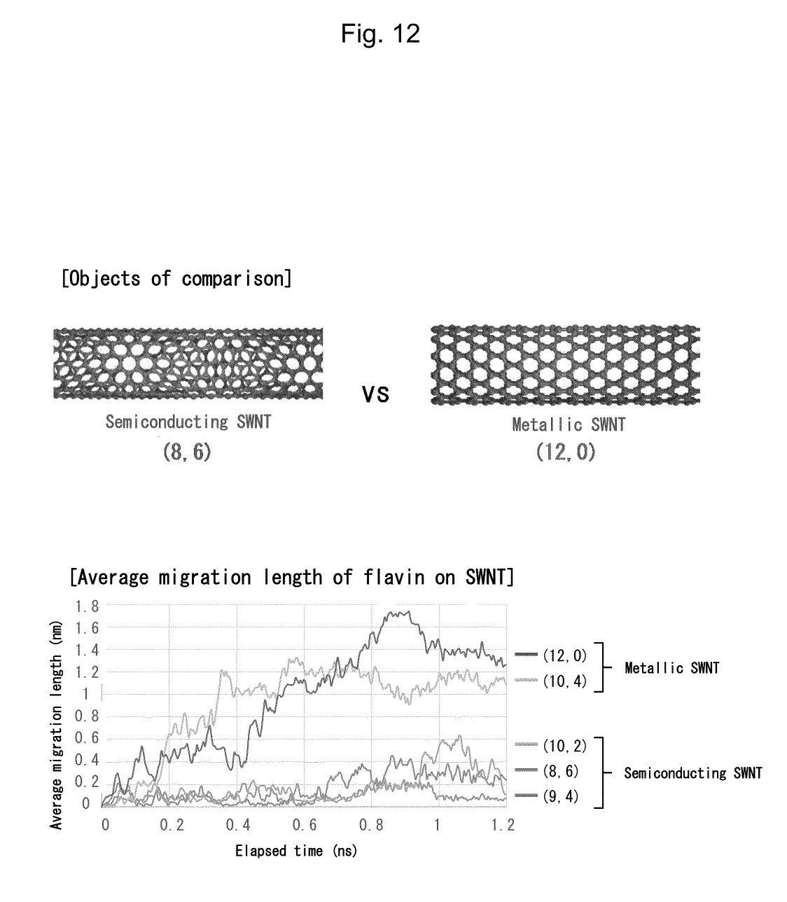

Interactions (adsorptive properties) of flavin derivatives (FC12 or dmC12) (specifically, dimers formed between flavin derivatives) with the semiconducting SWNTs and the metallic SWNTs were compared by measuring the average migration lengths of the flavin derivatives on the respective SWNTs according to the following measurement and experiment conditions. Specifically, the MD (Molecular Dynamics) was carried out following structural optimization with the molecular mechanics calculation (MM). The results are shown in FIG. 12.

<Measurement and Experiment Conditions>
MM (molecular mechanics calculation): SCHRODINGER MACROMODEL 9.6
MD (Molecular Dynamics): Desmond
Molecular force field: OPLS 2005
Solvent: toluene
Temperature: 300 K
Time: 1.2 ns
28 flavin derivative dimers were placed on respective SWNTs ((8,6) SWNT (semiconducting SWNT) and (12,0) SWNT (metallic SWNT))

As can be appreciated from the results shown in FIG. 12, the average migration lengths of the flavin derivative dimers on the respective SWNTs were significantly shorter for the semiconducting SWNTs than the metallic SWNTs. Accordingly, the flavin derivative dimer was found to have significantly greater interaction with the semiconducting SWNTs (significantly greater adsorptive property with the semiconducting SWNTs) than with the metallic SWNTs, and that found to be capable of selectively solubilizing the semiconducting SWNTs.

### Example 8

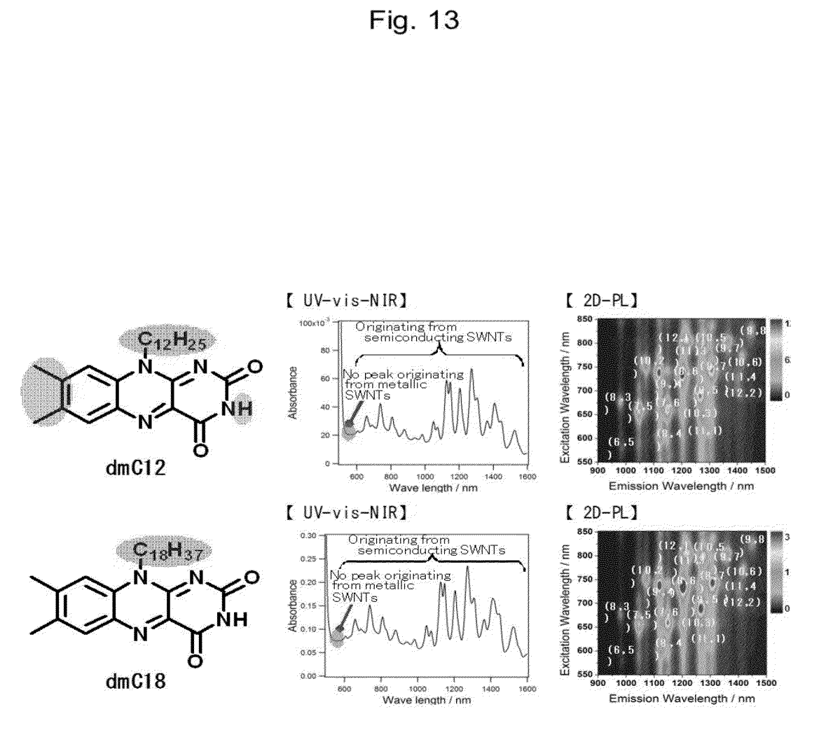

Following the procedure and the method of Example 1, an absorption spectrum and a photoluminescence spectrum of SWNTs dispersed in toluene with one of flavin derivatives, 10-octadecyl-7,8-dimethyl-10H-benzo[g]pteridine-2,4-dione (dmC18) were determined in the same manner as the absorption spectrum and the photoluminescence spectrum of the SWNTs dispersed in toluene with dmC12 (FC12) (see FIGS. 1 and 2). The absorption spectrum (UV-vis-NIR) and the photoluminescence spectrum (2D-PL) of the SWNTs obtained with dmC18 are shown in FIG. 13.

As a result, similar to the case using dmC12 (FC12), the semiconducting SWNTs were confirmed to be selectively solubilized among the SWNTs when dmC18 was used.

### INDUSTRIAL APPLICABILITY

Since the present invention is capable of obtaining SWNTs that have been separated into semiconducting SWNTs and metallic SWNTs in a single working step with an inexpensive equipment, the present invention is extremely beneficial in terms of usability. The present invention is also capable of obtaining SWNTs with longer lengths at a high recovery rate as compared to conventional techniques. Furthermore, according to the present invention, since the dispersant can be removed after separating the semiconducting SWNTs and the metallic SWNTs, application to a wide range of usage is not restrained by separation. Therefore, the present invention is also extremely beneficial in terms of practical use.

1. A method for separating a metallic single-walled carbon nanotube and a semiconducting single-walled carbon nanotube from single-walled carbon nanotubes, said method comprising the steps of:
dispersing the single-walled carbon nanotubes in a solution containing a low-molecular-weight compound having an alkyl chain moiety for exhibiting solubility in a solvent and an aromatic-ring-containing moiety for interacting with the single-walled carbon nanotubes; and separating said dispersion solution into a solution fraction and a solid fraction.

2. The method according to claim 1, wherein the low-molecular-weight compound comprises a flavin derivative.

3. The method according to claim 2, wherein the flavin derivative comprises 10-dodecyl-7,8-dimethyl-10H-benzo pteridine-2,4-dione and/or 10-octadecyl-7,8-dimethyl-10H-benzo[g]pteridine-2,4-dione.

4. The method according to any one of claims 1-3, wherein the solution fraction comprises the solubilized semiconducting single-walled carbon nanotube while the solid fraction comprises the metallic single-walled carbon nanotube.

5. The method according to claim 1, wherein the dispersion is carried out by stirring, shaking, ball milling or ultrasonic irradiation.

6. The method according to claim 1, wherein the separation is carried out by settling, filtration, membrane separation, centrifugation or ultracentrifugation.

7. The method according to claim 1, further comprising the steps of: collecting the semiconducting single-walled carbon nanotube from the above-described solution fraction; and/or collecting the metallic single-walled carbon nanotube from the above-described solid fraction.

8. An agent for separating a metallic single-walled carbon nanotube and a semiconducting single-walled carbon nanotube, the agent comprising a low-molecular-weight compound having an alkyl chain moiety for exhibiting solubility in a solvent and an aromatic-ring-containing moiety for interacting with the single-walled carbon nanotubes.

9. The separating agent according to claim 8, wherein the low-molecular-weight compound comprises a flavin derivative.

10. The separating agent according to claim 9, wherein the flavin derivative comprises 10-dodecyl-7,8-dimethyl-10H-benzo[g]pteridine-2,4-dione and/or 10-octadecyl-7,8-dimethyl-10H-benzo[g]pteridine-2,4-dione.
